(19) United States
(12) Patent Application Publication
(10) Pub. No.: US 2018/0042874 A1
(43) Pub. Date: Feb. 15, 2018

(54) METHOD FOR TREATING OSTEOPOROSIS

(71) Applicant: China Medical University, Taichung City (TW)

(72) Inventors: YI-YING WU, Taichung City (TW); I-CHEN YANG, Hsinchu City (TW); Chun-Hao Tsai, Taichung City (TW); PO-HAO HUANG, Taichung City (TW)

(73) Assignee: China Medical University, Taichung City (TW)

(21) Appl. No.: 15/675,826

(22) Filed: Aug. 14, 2017

(30) Foreign Application Priority Data
Aug. 15, 2016 (TW) 105125927

(51) Int. Cl. A61K 31/17; A61K 9/00

(52) U.S. Cl. CPC A61K 31/17; A61K 9/0053

(57) ABSTRACT

A method for treating osteoporosis in a subject in need thereof comprising administering to the subject an effective amount of a composition comprising a compound of formula I or pharmaceutically acceptable salts thereof.

### CROSS-REFERENCES TO RELATED APPLICATIONS

The present application relies in Taiwan Application No. 105125927, flied on Aug. 15, 2016, for priority, which is hereby incorporated by reference in its entirety.

### FIELD OF THE INVENTION

The present invention is related to a method for treating osteoporosis by a compound of formula I or pharmaceutically acceptable salts thereof.

### BACKGROUND OF THE INVENTION

Bone not only allows us to have motor function, but also helps us generate blood cells, store minerals and regulate pH values in our bodies. Osteocalcin, a complex protein of osteoblast, enhances calcification of the osteoblast to form bone, is a marker for osteogenesis. The balance of bone remodeling and bone resorption is also regulated by hormones. Bone remodeling is an initiation of bone turnover in order to maintain bone strength and mineral homeostasis. During bone remodeling, osteoclasts and osteoblasts are the leading factors of the resorption of old bones and the formation of new bones, which prevents the accumulation of bone microdamages. The bone remodeling rate increases at menopause and early onset menopause in female individuals, because the bone remodeling rate is increased, aging slows down, however the aging rate is still faster than that of average individuals. Osteoclasts, derived from hematopoietic stem cells and mesenchymal stem cell, are mainly responsible for bone resorption while osteoblasts are responsible for bone formation and mineralization. These two types of cells help our bodies to continuously provide calcium, magnesium, phosphorus, which are crucial materials for the formation of healthy bones. Normal aging process and menopause-related estrogen loss increase the risk of osteoporosis in women. However, when estrogen is deficient, the entire regulatory procedure is interrupted, the production of osteoblasts is reduced and the production of osteoclasts is increased, resulting in an abnormal increase in bone resorption. Estrogen loss for 5-8 years will accelerate the loss of bone density. In addition to estrogen loss, there are other factors that increase the risk of osteoporosis in women. Irreversible factors include age, gender, race, family history of osteoporosis, previous fractures, etc. Bone weakening results in an increased risk of bone fracture which often occurs in hips, spines and wrists.

Osteoprogenitor cells maintain the production of osteoblasts from which new bone matrix is synthesized and new bone is formed on the surface of the new bone matrix. Osteocytes are inside the bone matrix to support the bone structure and cover the surface of static bone to provide a protective effect. Osteoblast lineage regulates by responding to various types of hormones, mechanisms and cytokines. Osteoblasts are usually stimulated by parathyroid hormone (PTH), prostaglandin E2 (PGE2), triiodothyronine (T3), tetraiodothyronine (T4), transforming growth factor-β (TGF-β) and 1,25-dihydroxyvitamin D (1,25 (OH) 2D3) and regulated by glucocorticoid, they react differently in different situations. In cases of primary hyperthyroidism, although osteoblasts have sex hormone receptors and may carry transforming growth factor-β and collagen when stimulated by sex steroids in vitro, there is no direct evidence to show how they act in vivo.

In addition, osteoblasts can synthesize and secrete some growth factors, such as platelet derived growth factor (PDGF), insulin-like growth factors (IGFs), transforming growth factor-β, endothelin-1, etc. They can also secrete by themselves to impose effects on the cells themselves. When differentiation process is normal, pericytes from peripheral blood (blood vessel pericytes) can develop into osteoblast phenotype. The transformation of mesenchymal stem cells into osteoblast lineages requires the typical Wnt/β-catenin pathway and associated proteins. The Wnt signaling pathway plays an important role in regulating the formation of cartilage, the formation of hemocytes, and possible stimulation or inhibition of osteoblast differentiation in various stages. When differentiating into an osteoblast, the morphology of osteoblast precursor cells transforms from spindle-like osteoprogenitor cells into a large cubic shape of osteoblasts. As to bone remodeling, usually the function of osteoblasts is the ability to detect the expression of alkaline phosphatase. An activated mature osteoblast synthesizes bone matrix, possesses a larger nucleus, expands the structure of the Golgi apparatus and a massive endoplasmic reticulum. It also secretes osteocalcin (BGP), a large amount of type I collagen (about 90%) and other matrix proteins, for example: osteonectin and osteopontin.

Osteoclast cells are currently the only known cells that undertake bone resorption. Active, multinucleated osteoclast cells are derived from the mononuclear precursor cells monocyte/macrophage. After being evaluated histologically, the mononuclear precursor cells of monocytes/macrophages in born marrow are more likely to develop into osteoclasts. It has been proved that many growth factors and cytokines which involve in inflammatory responses and rheumatic diseases also affect the differentiation and function of osteoclasts, or directly affect osteoclast lineage, or indirectly affect other types of cells, crucially regulate the expression of receptor activator of nuclear factor-κB ligand (RANKL), or inhibit osteoprotegerin (OPG) of the osteoclasts. Receptor activator of nuclear factor-κB ligand and macrophage colony stimulating factor (M-CSF) are the primary factors of osteoclast formation. The diameter of an osteoclast cell is about 20-50 mm, contains 1-5 nuclei, when its activity is enhanced because of the stimulation of the parathyroid hormone (PTH), the number of nuclei increases to 20 or more. When an osteoclast cell initiates the osteolytic mechanism, it forms abnormal folds on the edge and with proton pumps inside to release H⁺ out of the cell to create an acidic external environment and produces enzymes (acid phosphatase, proteases and lysosomal enzymes). Osteoclast maturation is stimulated by IL-1, IL-6, TGF-α, TNF, lymphotoxin, and parathyroid hormone-related protein (PTH-rP). Therefore, these materials may be secreted due to some benign or malignant diseases, enhancing osteolysis and causing bone loss and osteoporosis.

Members of tumor necrosis factor (TNF) family of protein and members of tumor necrosis factor receptor (TNFR) family of proteins play very important roles, they control cell death, proliferation, autoimmune, immune cell function or lymphoid organ cell formation. Recently, the key function of the new members of this large family in immunization has been identified, the function is coupled with lymphocytes and other systemic organs, for example: bone remodeling and mammary gland formation during pregnancy. Bone remodeling causes resorption of old bone by osteoclasts and formation of new bone by osteoblasts simultaneously. The regulation of bone remodeling is mediated through a variety of mechanisms, which ultimately lies on the interaction among osteoclasts or the interaction among their precursor cells, osteoblasts and bone marrow-derived stem cells. Osteoblasts and osteoclasts are derived from different cell lineages and maturation processes, i.e., osteoblasts are derived from mesenchymal stem cells while osteoclast cells are differentiated from hematopoietic monocyte/macrophage precursor cells.

Members of tumor necrosis factor family-receptor activator of nuclear factor-κB ligand (also known as OPGL, TRANCE, ODF and TNFSF11) and its receptor-receptor activator of nuclear factor κB (RANK) are key to bone resorption, and essential to the regulation of osteoclast activation and maturation. Receptor activator of nuclear factor-κB ligand induces the differentiation of osteoclast precursor cells and stimulates the function of resorption and the survival of mature osteoclasts. Tumor necrosis factor receptor-associated factors (TRAFs) adaptor proteins play an important role when the receptor activator of nuclear factor κB signaling pathway begins. TNF receptor-associated factor protein is the most important factor of receptor activator of nuclear factor κB signaling in osteoclast cells. This protein can transmit receptor activator of nuclear factor κB signals to downstream targets, which include: nuclear factor-κB (NF-κB) and c-JUN N-terminal kinase (INK). NF-κB is retained in the cytoplasm and acts as a protein complex which inhibits I-κB (inhibitor of kappa light chain gene enhancer in B-Cells) in unstimulated cells. When NF-κB activation is stimulated, it induces the activation of I-κB kinase (IKKs), resulting in phosphorylation, followed by proteasome regulation of I-κB degradation. NF-κB is released and then enters the nucleus to bond to the DNA target position. Receptor activator of nuclear factor κB stimulates differentiated osteoclast cells or osteoclast precursor cells, including phosphorylation, I-κB-α degradation, nuclear translocation, and bonding of NF-κB protein p50, p52 and p62 to DNA. Other adaptor proteins in the cell will interact with TNF receptor-associated factor proteins and regulate the function of TAK1 (TGF-beta activated kinase) and NIK (NF-κB-Inducing Kinase) of the tumor necrosis factor receptor-associated factor in osteoclasts. XIAP (Xenopus Inhibitor of Apoptosis) and cIAP (Cellular Inhibitors of Apoptosis) are NF-κBs, which are downstream targets of the osteoclast survival pathway. All three members MAPK (mitogen activated protein kinase) family, ERK (extracellular signal regulated kinase), osteoclasts or osteoclast precursor cells, activate JNK and p38 via receptor activator of nuclear factor κB. JNK1 is very important in affecting osteoclast differentiation. JNK upstream targets include MKK7 (mitogen-activated protein kinase kinase 7). Dominant-negative MKK6 (mitogen-activated protein kinase kinase 6) regulates p38 activity and osteoclast differentiation. Raf involves with ERK signal transduction, participates in the activation of CD40 which is a receptor of tumor necrosis factor receptor (TNFR) family, the signaling characteristics of which are similar to those of receptor activator of nuclear factor κB. Activation of the downstream receptor activator of nuclear factor κB signaling pathway of the tumor necrosis factor receptor-associated factor 6 (TRAF6) will induce JNK and p38 activation. ERK and JNK induce downstream target AP-1 (activating protein-1) which is comprised of dimer protein c-Fos and c-Jun. ERK induces and activates c-Fos and NFAT (nuclear factor of activated T-cells) by phosphorylating the part of Elk1 that belongs to TFC (ternary complex factor), while JNK increases the transcription activity of AP-1 by phosphorylating c-Jun.

Src acts as a modulator of receptor activator of nuclear factor κB in PI3K (phosphatidylinositol-3 kinase)/Akt1 signaling. In so many downstream molecules of Src, protein tyrosine kinase-2 (PYK2) and c-Cbl are the ones that involved with adsorption signaling and bone resorption function of the osteoclasts. In addition, osteoclasts are able to form important actin filaments because of the association between actin-binding protein gelsolin and PI3K. The function of Src and PI3K depends on the fusion of receptor activator of nuclear factor κB and adsorption signaling, correct signals are transmitted to actin and cytoskeleton, which is beneficial to osteoclast activation and resorption.

An increased activity of the osteoclasts is common in many osteopenic diseases, including postmenopausal osteoporosis, Paget's Disease, lytic bone metastases or rheumatoid arthritis (RA), leading to an increased bone resorption and severe bone damages. The function of receptor activator of nuclear factor κB ligand is inhibited by a natural decoy receptor osteoprotegerin to prevent postmenopausal osteoporosis and cancer metastasis and to provide an excellent therapeutic effect on osteoporosis.

Osteoporosis often occurs in middle-aged people and post-menopausal women, currently there are more than 5 million people in Taiwan who are at risk of developing osteoporosis, it may go up to 7.5 million by 2020, patients with osteoporosis are susceptible to bone fractures, the medical and social costs for patient care and treatment are tremendous. At present, therapeutic drugs for osteoporosis often have side effects, or they are ineffective, or very expensive, therefore on skilled in the art is looking for a compound which can treat osteoporosis with little side effect and inexpensive.

### BRIEF DESCRIPTION OF THE DRAWINGS

FIG. 1 shows the test of cytotoxicity of the compound of formula I in RAW 264.7 cells. A is cell survival rate obtained by using MTT detection method. RAW 264.7 cells are cultured in different concentrations of the compound of formula I, after 72 hours MTT reagent is added to each well, detection is made based on the light absorption value at 550 nm, cell survival rate is then calculated. The results are the average of three experiments; the Microsoft Office Excel is used to calculate ±SD of the average values, and the differences in data are evaluated by using the Student's t-test; * p<0.05, ** p<0.005. B is cell survival rate obtained by using MTT detection method. RAW 264.7 cells are treated with 10 μg/ml, 20 μg/ml of the compound of formula I, after being cultured for 3, 6, 9, and 12 days, MTT reagent is added into each well, detection is made based on the light absorption value at 550 nm, cell survival rate is then calculated. The results are the average of three experiments.

FIG. 2 shows the effect of different concentrations of the compound of formula I on the induction of osteoclast cells by receptor activator of nuclear factor κB ligand. A shows RAW 264.7 cells, cultured in a 24-well plate, each well contains 1×10⁴ cells, treated with 100 ng/ml of receptor activator of nuclear factor κB ligand, or co-treated with 10 μg/ml, 20 μg/ml of the compound of formula I for 6 days. After being stained with tartrate resistant acid phosphatase, observe with a 400× microscope. B shows tartrate resistant acid phosphatase stain-positive multinucleated cells containing three nuclei or multiple nuclei, they are counted as osteoclast cells. The results are the average of three experiments; the Microsoft Office Excel is used to calculate ±SD of the average values, and differences in data are evaluated by using the Student's t-test; ** p<0.005.

FIG. 3 shows the effect of the compound of formula I, 10 μg/ml and treated for different time periods, on osteoclasts induced by receptor activator of nuclear factor κB ligand. A shows RAW 264.7 cells, cultured in a 24-well plate, each well contains 1×104 cells, treated with 100 ng/ml of receptor activator of nuclear factor κB ligand or co-treated with 10 μg/ml of the compound of formula I for 2, 4, 6 days. After being stained with tartrate resistant acid phosphatase, observe with a 400× microscope. B shows tartrate resistant acid phosphatase stain-positive multinucleated cells containing three nuclei or multiple nuclei, they are counted as osteoclast cells. The results are the average of three experiments; the Microsoft Office Excel is used to calculate ±SD of the average values, and differences in data are evaluated by using Student's t-test; *p<0.05, ** p<0.005.

FIG. 4 shows that the compound of formula I affects the activation of MAPK proteins induced by receptor activator of nuclear factor κB ligand in osteoclast cells. A shows that RAW 264.7 cells are treated with 100 ng/ml receptor activator of nuclear factor κB ligand or co-treated with 10 μg/ml of the compound of formula I, proteins are collected at different time points and the protein expression of ERK, p38, JNK in the MAPK pathway are detected by using the Western blot method. B shows the protein expression of ERK, p38, JNK in the MAPK pathway detected by using the Western blot method, the expression are quantified by using the image J, the results are the average of three experiments; the Microsoft Office Excel is used to calculate ±SD of the average values, differences in data are evaluated by using the Student's t-test; ** p<0.005.

FIG. 5 shows that the compound of formula I affect the activation of MAPK downstream proteins and NF-κB induced by receptor activator of nuclear factor κB ligand in osteoclast cells. A shows that RAW 264.7 cells are treated with 100 ng/ml of receptor activator of nuclear factor KB ligand or co-treated with 10 μg/ml of the compound of formula I for 24, 48 hours. B shows the protein expression of the downstream protein molecules NFATc2 and c-Fos in the MAPK pathway and NF-κB detected by using the Western blot method, the expression are quantified by using the image J, the results are the average of three experiments; the Microsoft Office Excel is used to calculate ±SD of the average values, differences in data are evaluated by using the Student's t-test; ** p<0.005.

FIG. 6 shows that the compound of formula I affect the osteolytic activity of osteoclast cells induced by receptor activator of nuclear factor κB ligand. A shows that RAW 264.7 cells are treated with 100 ng/ml of receptor activator of nuclear factor κB ligand or co-treated with 10 μg/ml of the compound of formula I, cultured in a corning osteo assay surface culture plate for 12 days, then observe with a 40×microscope. B shows that the compound of formula I inhibits the bone resorption activity of osteoblast cells; the results are the average of three experiments; the Microsoft Office Excel is used to calculate ±SD of the average values, and differences in data are evaluated by using the Student's t-test; **p<0.005. The range of bone resorption is quantified by using Image J.

FIG. 7 shows the effect of various treatments on the body weight of rats, A is the body weight of the rats, and B is the relative body weight of the rats.

FIG. 8 shows the effect of various treatments on the bone density of the lumbar spine and tibia of rats, A is the relative bone density of the lower lumbar spine (the third to the fifth lumbar vertebrae, L3-L5), and B is the relative bone density of the tibia of rats.

FIG. 9 shows the effect of various treatments on liver and kidney toxicity in rats, A is the effect of various treatments on alanine aminotransferase in rats; B is the effect of various treatments on blood urea nitrogen in rats; and C is the effect of various treatments on plasma creatinine in rats.

### SUMMARY OF THE INVENTION

The present invention provides a method for treating osteoporosis in a subject in need thereof comprising administering to the subject an effective amount of a composition comprising a compound of formula I or pharmaceutically acceptable salts thereof, wherein the compound of formula I is as follows:

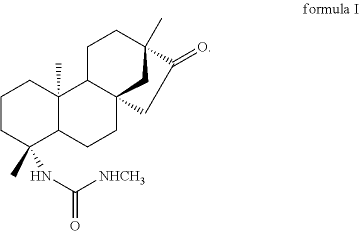 [SMILES: CNC(=O)N[C@]1(C)CCC[C@@]2(C)C3CC[C@@]4(C)C[C@]3(CCC21)CC4=O]

### DETAIL DESCRIPTION OF THE INVENTION

Unless otherwise specified, “a” or “an” means “one or more”.

Bone remodeling is a brief regulatory process which causes simultaneous bone resorption and bone formation. Osteoclasts and osteoblasts are the ones mostly involved in this process.

Bone remodeling begins when osteoclastic bone resorption is completed, which takes about 3-5 weeks, the surface of the reabsorbed site attracts osteoblasts, multiple mononuclear osteoblasts fill the absorbed site to form new matrix, the osteoblast cells then remodel the bone matrix and cause mineralization, which takes 3-5 months to complete.

Some biologically active and important compounds may exist in certain specific sites of natural plants, for example, paeonol reduces osteoclast differentiation by inhibiting ERP, P38, NF-κB signaling pathways; in addition, pyrroloquinoline quinine and coptisine also have inhibitory effect on osteoclast differentiation. These compounds can prevent bone loss and treat osteoporosis by inhibiting excessive osteoclast formation.

It has been proved that 1, 25-dihydroxyvitamin D3 (Vitamin 1,25 (OH)₂D₃), estrogens, androgens, TGF-α, IGF I/II are capable of enhancing osteocyte differentiation in vitro. The activity of alkaline phosphatase and the production of osteocalcin have also been identified.

Stevioside is a natural sweetener that exists in the leaves of stevia rebaudiana bertoni, it is a calorie-free sugar substitute used in a wide range of food and beverages. In general, natural compounds are often used as molecular templates for manufacturing novel drugs. Isosteviol (ent-16-oxobeyeran-19-oic acid) is an ent-beyerane tetracyclic diterpene, a compound obtained through acid hydrolysis of stevioside. Previous studies have shown that after being chemically modified isosteviol-derived compounds exhibit a variety of biological activities, including: anti-hypertension, anti-inflammation, anti-diarrhea, anti-tumor, inhibition of bactericidal activities and immunoregulatory functions. In addition, isosteviol derivatives exhibit significant inhibitory effects on the activation of early antigens of Epstein-Barr virus (EBV).

The chemical formula of the compound of formula I is: ent-16-oxobeyeran-19-N-methylureido, the molecular weight is 346.51, prepared from a number of isosteviol derivatives by substituting a Ureide group with a COOH group on the 19th carbon. Recent studies showed that the compounds of formula I can effectively suppress the secretion of hepatitis B virus (HBV) surface antigen (HBsAg) and hepatitis B virus antigen (HBeAg) when human hepatocellular carcinoma cells are used as a research platform. Osteoclast formation and activation are associated with several different key protein kinases signaling pathways; these protein pathways are NF-κB kinase, JNK, p38, ERK. Therefore, the present invention has developed a drug of natural origin for the treatment of osteoporosis, the compound of formula I of the present invention has been developed as a drug for treating osteoporosis with very little side effect.

The present invention provides a method for treating osteoporosis in a subject in need thereof comprising administering to the subject an effective amount of a composition comprising a compound of formula I or pharmaceutically acceptable salts thereof, wherein the compound of formula I is as follows:

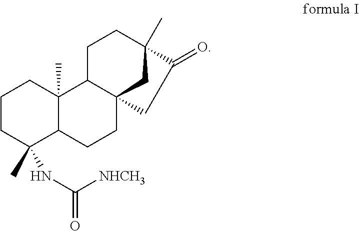 [SMILES: CNC(=O)N[C@]1(C)CCC[C@@]2(C)C3CC[C@@]4(C)C[C@]3(CCC21)CC4=O]

According to the present invention, in a preferred embodiment, the composition inhibits osteoclast differentiation; in another preferred embodiment, the composition inhibits osteolytic activity of osteoclasts.

According to the present invention, in a preferred embodiment, the dose of the compound of formula I is 1 mg/kg to 400 mg/kg; in another preferred embodiment, the dose of the compound of formula I is 1 mg/kg to 80 mg/kg; in a more preferred embodiment, the dose of the compound of formula I is 1 mg/kg to 25 mg/kg and the composition is an oral dosage form. For dosage calculations for human and various animals, please refer to http://www.taiwanbio.com.tw/property_detail.php?sno_property=62, doses described here may be adjusted according to the animal to be treated.

### Examples

The examples of the present invention will be described in detail below, please refer to the drawings and the detailed description at the same time. The examples below are non-limiting and are merely representative of various aspects and features of the present invention.

The method of preparing the compound of formula I can be found in Phytochemistry 2014, 99: p. 107-114, the structure of which is as follows:

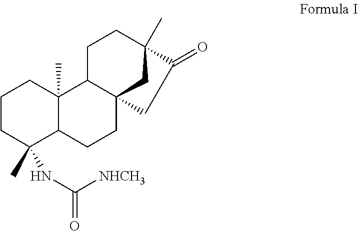 [SMILES: CNC(=O)N[C@]1(C)CCC[C@@]2(C)C3CC[C@@]4(C)C[C@]3(CCC21)CC4=O]

### Cell Culture

The present invention used monocyte/macrophage cell line RAW 264.7 from mouse as an experimental model of the osteoclast differentiation system. RAW 264.7 cells differentiated into osteoclasts in an environment containing receptor activator of nuclear factor κB ligand. RAW 264.7 was obtained from a tumor induced by Abelson murine leukemia virus and purchased from the American Type Culture Collection (ATCC; Rockville, Md.). To culture osteoclasts, RAW 264.7 cells were used, cultured in a 24-well plate, the number of cells in each well was 1×10⁴ cell/ml and cultured in a medium containing 100 ng/ml of receptor activator of nuclear factor κB ligand or co-treated with different concentrations of the compound of formula I.

### Cytotoxicity Test

MTT (3-(4,5-dimethylthiazol-2-yl)-2,5-diphenyl tetrazolium bromide is yellow when dissolved in a phenol red-free medium. The yellow MTT solution is reduced by succinate dehydrogenase in mitochondria of living cells into purple insoluble formazan crystals, deposited in the cells generating blue formazan crystals. The amount of formazan crystals produced is proportional to the number of viable cells (succinate dehydrogenase in dead cells is degraded and unable to reduce MTT). Formazan crystals can be dissolved in acidic isopropanol or dimethyl sulfoxide (DMSO), and an enzyme linked immunosorbent assay detector can be used to measure the light absorption value, therefore, MTT can be used as a marker for cell survival rate.

Cytotoxicity test of the compound of formula I in RAW 264.7 cells was studied and detected by using the MTT colorimetric assay. Firstly, RAW 264.7 cells were cultured in a 24-well plate, the cell number in each well was 2×10⁴ cell/ml, after being treated with a culture solution containing 0, 1, 5, 10, 20, 40, 80 μg/ml of the compound of formula I for 3 days the culture solution was removed, and the appropriate concentration for subsequent experiments was confirmed. Secondly, to confirm that no toxicity would be produced by the compound of formula I during the designed testing period, RAW 264.7 cells were cultured in a 24-well plate, the cell number in each well was 2×10³ cell/ml, the selected concentrations of the compound of formula I were 0, 10, 20 μg/ml and the cells were treated for 3, 6, 9, 12 days, the culture solution was replaced with fresh one of the same condition once every three days. After reaction was completed, the MTT culture solution was prepared in a 10-fold dilution manner, i.e., 1 ml of the culture solution containing 0.1 ml 5 mg/ml of MTT and 0.9 ml of FBS-free DMEM broth. After being cultured in an incubator at 37° C. for 4 hours, equivalent amount of cell culture solution by volume was added into dimethyl sulfoxide to dissolve MTT Formazan, 100 μl was respectively collected and placed in a 96 well-plate, and the light absorption value at 570 nm was determined by using an enzyme immunosorbent assay detector. The Cell survival rate (%) was calculated by using the following formula: (light absorption value of drug-treated cells)/light absorption value of culture solution-treated cells)×100%.

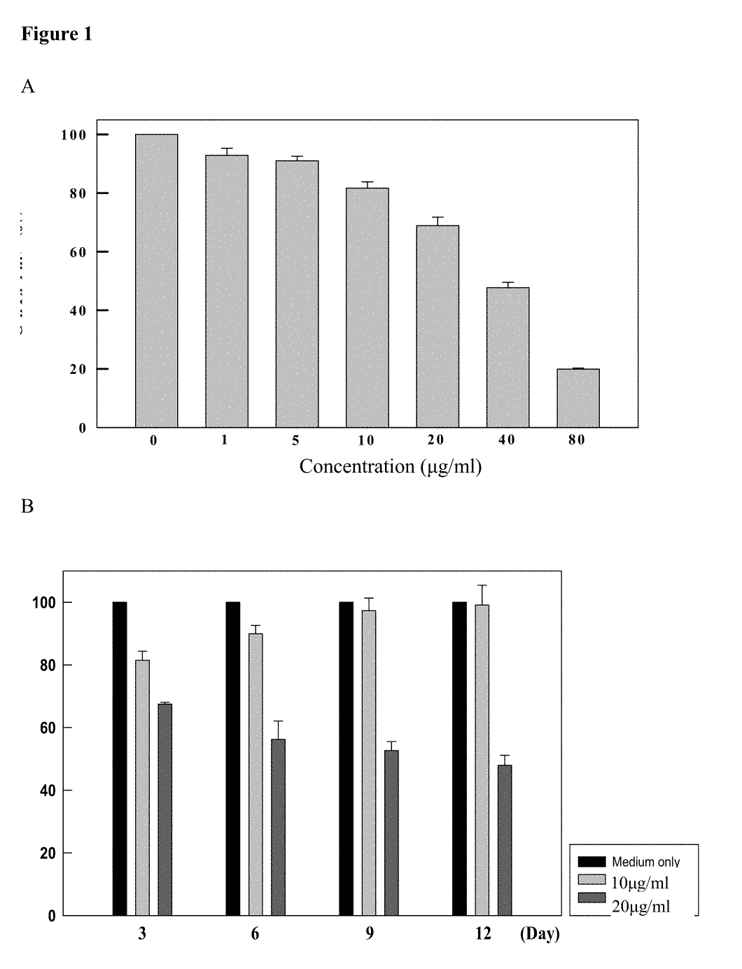

Firstly, we would like to know whether the compound of formula I were toxic to RAW 264.7 cells, and concentrations appropriate for subsequent experiments were to be selected. After being treated with 0, 1, 5, 10, 20, 40, 80 μg/ml of the compound of formula I for 72 hours, detected with the MTT colorimetric assay. The results showed that IC50 for the compound of formula I was at a concentration of about 40 μg/ml. The compound of formula I was cytotoxic when the concentration was higher than 40 μg/ml which caused a decreased survival rate of RAW 264.7 cells (FIG. 1A), therefore concentrations lower than 40 μg/ml were selected for subsequent experiments. To confirm the selected concentrations of the compound of formula I would not produce cytotoxin in the subsequent experiments, 10 μg/ml, 20 μg/ml of the compound of formula I were respectively used for treatment of 3, 6, 9, 12 days, the culture solution was replaced with fresh ones of the same condition once every three days and then detected with the MTT colorimetric assay. The data showed that after being treated with 10 μg/ml of the compound of formula I, as time goes by no cytotoxin was produced, therefore this concentration was selected for subsequent experiments (FIG. 1B).

### Osteoclast Differentiation Test

In order to simulate physiological functions of the bone, we simulated the in vivo environment and some hormones essential to differentiation were added. The hormone environment suitable for osteoblast cells was simulated for osteoclast differentiation. Mononuclear osteoclasts transformed into polynuclear osteoclasts through membrane fusions, and the osteoclasts were activated, the edge became folded and the time required for osteoclast maturation was about 6 days which could be divided into two stages, the first four days were the period for the proliferation of osteoclast precursor cells, the last two days were the period for osteoclast differentiation. Each stage required stimulation from different cytokines or nutrients, for example: macrophage colony-stimulating factor (M-CSF) was required for proliferation, macrophage colony-stimulating factor and receptor activator of nuclear factor κB ligand were required for differentiation. In addition, different stages of osteoblast differentiation exhibited respectively specific biochemistry, such as tartrate resistant acid phosphatase (TRAP), calcitonin receptors (CTR), etc. Markers for osteoclast differentiation were evaluated by staining with tartrate-resistant acid phosphatase. Tartrate-resistant acid phosphatase was located in the folds at the edge of the osteoclast, when the bone reabsorbed the tartrate-resistant acid phosphatase was released from the cell. Thus, osteoclast differentiation could be indicated by detecting the activity of tartrate-resistant acid phosphatase.

Acid phosphatase was capable of catalyzing the hydrolysis of phosphate monoester phosphoric acid in vitro at pH 4-6. When monoester phosphoric acid substrate Naphthol AS-BI phosphate was added by this method, acid phosphatase would hydrolyze phosphoric acid; the residual Naphthol AS-BI together with diazonium and Fast garnet GBC salts formed an insoluble, color of red dates pigmentation. The activation site of acid phosphatase in osteoclasts could be dyed into color of red dates, a specific characteristic of the osteoclasts.

To observe the effect of the compound of formula I on osteoclast differentiation, RAW 264.7 cells were cultured in a 24-well plate, the cell number in each well was 1×10⁴ cell/ml, the culture solution contained 100 ng/ml receptor activator nuclear factor κB ligand (for RAW 264.7, Peprotech) or co-treated with 10, 20 μg/ml of the compound of formula I. After being treated for 6-7 days, stained with Tartrate-resistant acid phosphatase. In addition, the culture solution contained 100 ng/ml of receptor activator of nuclear factor κB ligand (for RAW 264.7, Peprotech) or co-treated with 10 μg/ml of the compound of formula I, after being treated for 2, 4, 6 days stained with tartrate-resistant acid phosphatase, osteoclast differentiation was then observed.

Osteoclast formation was detected by quantifying the staining positive rate of tartrate-resistant acid phosphatase (387-A, Sigma-Aldrich, St. Louis, Mo.). Samples were fixed with a fixing solution, naphthol AS-BI phosphate staining kit was used, the samples and the staining agent were allowed to react at 37° C. for 1 hour, and then stained with hematoxylin purple. The cells of the control group might be stained with positive red, therefore, tartrate-resistant acid phosphatase-positive multinucleated cells with three or more nuclei could be identified as osteoclasts and counted under an inverted phase contrast microscope. The method for counting multinuclear tartrate-resistant phosphatase-positive osteoclasts was to calculate the number of the formed multinuclear osteoclast cells in every 100 RAW 264.7 cells. The morphology of the osteoclast cells was further pictured for observation.

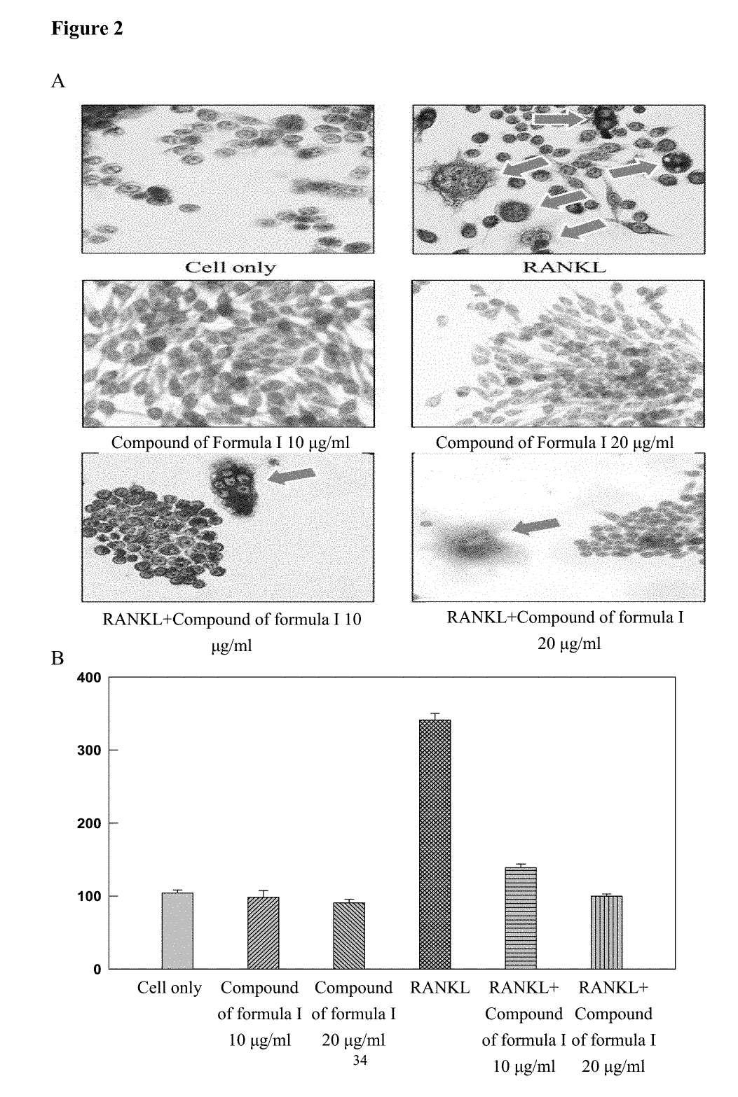

After the cytotoxicity of the compound of formula I in RAW 264.7 was understood, we used 100 ng/ml of receptor activator of nuclear factor κB ligand as the inducing substance for osteoclast differentiation, as the positive control group. In addition, the effect of the compound of formula I on osteoclast differentiation induced by receptor activator of nuclear factor κB ligand was observed after being co-treated with receptor activator of nuclear factor κB ligand and 10, 20 μg/ml of the compound of formula I, respectively for 6 days; 20 μg/ml of the compound of formula I was selected to avoid the situation that 10 μg/ml of the compound of formula I would not have any effect on osteoclast differentiation, so 20 μg/ml of the compound of formula I was selected for observation together. Tartrate-resistant acid phosphatase was used for special staining, then observed and counted with a microscope 400×, the results are shown in FIGS. 2A, 2B. The results showed that co-treatment of 100 ng/ml of receptor activator of nuclear factor κB ligand and 10 μg/ml of the compound of formula I resulted in a decrease in the level of osteoclast differentiation induced by receptor activator of nuclear factor KB ligand.

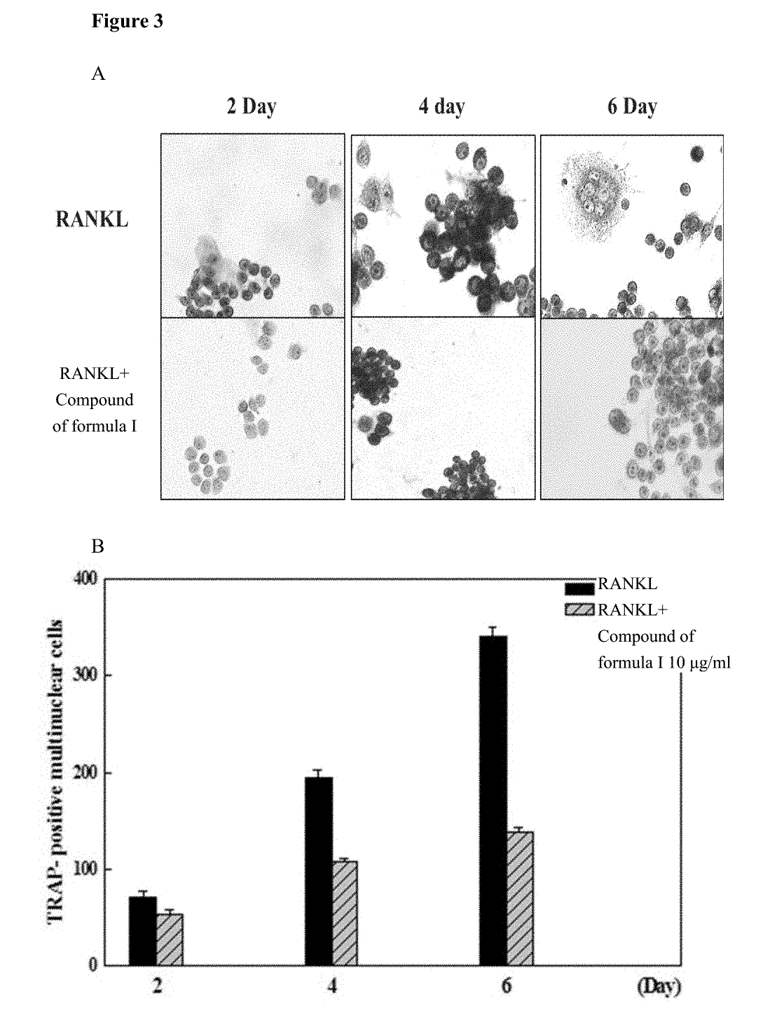

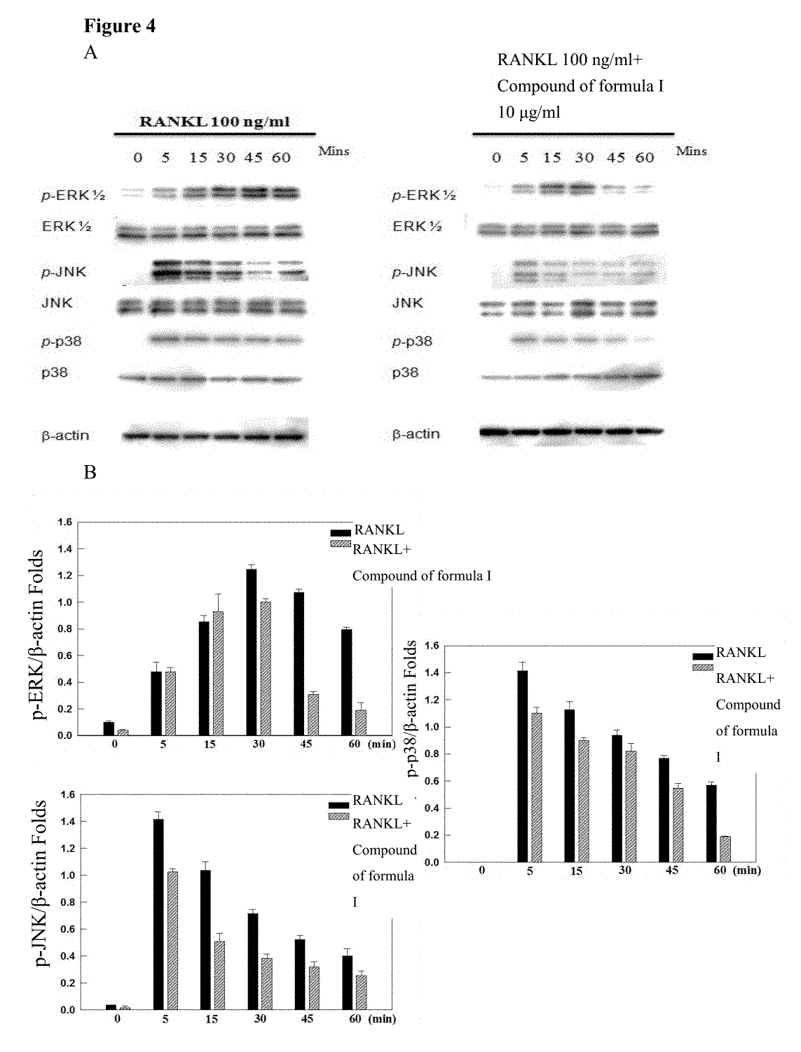

We selected 100 ng/ml of receptor activator of nuclear factor κB ligand and 10 μg/ml of the compound of formula I for treatment of 2, 4, 6 days respectively to confirm the effect of this concentration of the compound of formula I on the changes of the reduced level of osteoclast differentiation induced by receptor activator of nuclear factor κB ligand. Tartrate-resistant acid phosphatase was used for special staining, then observed and counted with a microscope 400×. The results showed that as time goes by the compound of formula I at a concentration of 10 μg/ml had a significant and inhibitory effect on osteoclast differentiation induced by receptor activator of nuclear factor κB ligand (FIGS. 3 A, 3B). Based on this result, in subsequent experiments co-treatment of 100 ng/ml of receptor activator of nuclear factor κB ligand and 10 μg/ml of the compound of formula I was used as the experimental group.

Effect of the compound of formula I on the pathways and conditions of osteoclast differentiation induced by receptor activator nuclear factor κB ligand. Periodic control of the cell transformation was investigated by using the Western blot method, protein signaling pathways in the cell often initiate different proteins, resulting in different cell responses. Previous scientific literature found that three MAPKs proteins, ERK (extracellular signal regulated kinase), p38 and JNK were able to regulate osteoclast differentiation. In addition, the present invention would also investigate MAPKs downstream proteins c-Fos and NFATc2, and observe NF-κB expression. Therefore, we would like to know whether the use of the compound of formula I would result in activation or inhibition of the aforementioned osteoclast-associated proteins and affect the operation of osteoclast differentiation signaling pathways.

The effect of the compound of formula I on the protein expression of osteoclast MAPKs pathways induced by receptor activator of nuclear factor κB ligand was studied, experiments were performed by using the Western blot method; RAW 264.7 cells were rinsed in ice-cold PBS, cell lysis buffer was added, the components of the buffer solution included: 10 mM 4-(2-hydroxyethyl)-1-piperazineethanesulfonic acid (HEPES), 1.5 mM MgCl₂, 0.5 mM dithiothreitol (DTT), 0.5 μg/ml leupeptin and 6.4% Nonidet P-40, reacted on ice for 15 minutes. The cells were scraped off with a spatula, collected into a 1.5 ml centrifuge tube, the cells were thoroughly broken by using a shaker; centrifuged at 12,000 rpm, 4° C. for 10 minutes; supernatant was added into a new 1.5 ml centrifuge tube; boiled with IX sodiumdodecyl sulfate (SDS) sample buffer, followed by electrophoresis using 10 to 12% SDS-PAGE. The molecular weight of the protein was separated by SDS-PAGE (sodium dodecyl sulfate/polyacrylamide gel electrophoresis); and then stain-transferred onto a PVDF membrane (polyvinylidene difluoride membrane). The PVDF membrane was then blocked with 5% skimmed milk powder in Tris-buffered saline containing 0.5% Tween-20 (TBST) for 1 hour, then reacted with primary rabbit: anti-phospho-ERK Ab, anti-phospho-JNK Ab (1:500, Cell Signaling), anti-ERK Ab, anti-JNK Ab, anti-p38 Ab (1:1000, Cell Signaling), anti-Anti-βactin Ab (1:10000, Cell Signaling) on a shaking platform at 50-60 rpm, 4° C. for 24 hours. The anti-NF-κB p65 Ab (1:1000, Cell Signaling) was then rinsed with TBST for three times, each time 15 minutes, the transfer membrane and horseradish peroxidase were bonded to anti-rabbit antibody (Santa Cruz Biotechnology) (Dilution 1:2000) for reaction at room temperature for one hour. The transfer membrane was rinsed again and the membrane loaded with chemical luminescence reagent ECL was allowed to react with a photosensitive sheet in a darkroom for a suitable period of time, the photosensitive sheet then reacted with the developer. After the photosensitive sheet was exposed to light a band-shaped antibody-binding proteins was observed, quantified by the density of the bands.

According to previous literature, RAW 264.7 cells differentiated into osteoclasts, most of which were induced by receptor activator of nuclear factor κB ligand, the osteoclast differentiation was accomplished by MAPK pathway or NF-κB pathway. The MAPK family included: ERK, p38, JNK which were key molecules for the differentiation of principal osteoclast cells. MAPK downstream molecules: c-Fos and NFATc2 also participated in osteoclast differentiation. In addition, we investigated NF-κB activity through NF-κB p65 protein expression.

RAW 264.7 cells were treated with 100 ng/ml of receptor activator of nuclear factor κB ligand or co-treated with 10 μg/ml of the compound of formula I to investigate whether the compound of formula I inhibited the activity of MAPK family members and NF-κB, had an effect on the reduced osteoclast differentiation. After the cells were treated for 0, 5, 15, 30, 45, 60 minutes, the proteins were collected for protein expression analysis by using the Western blot method.

As shown by the results, we were able to find ERK, p38, JNK (FIGS. 4A, B), when co-treated with receptor activator of nuclear factor κB ligand and the compound of formula I, the expression of phosphorylated ERK, p38, INK was decreased.

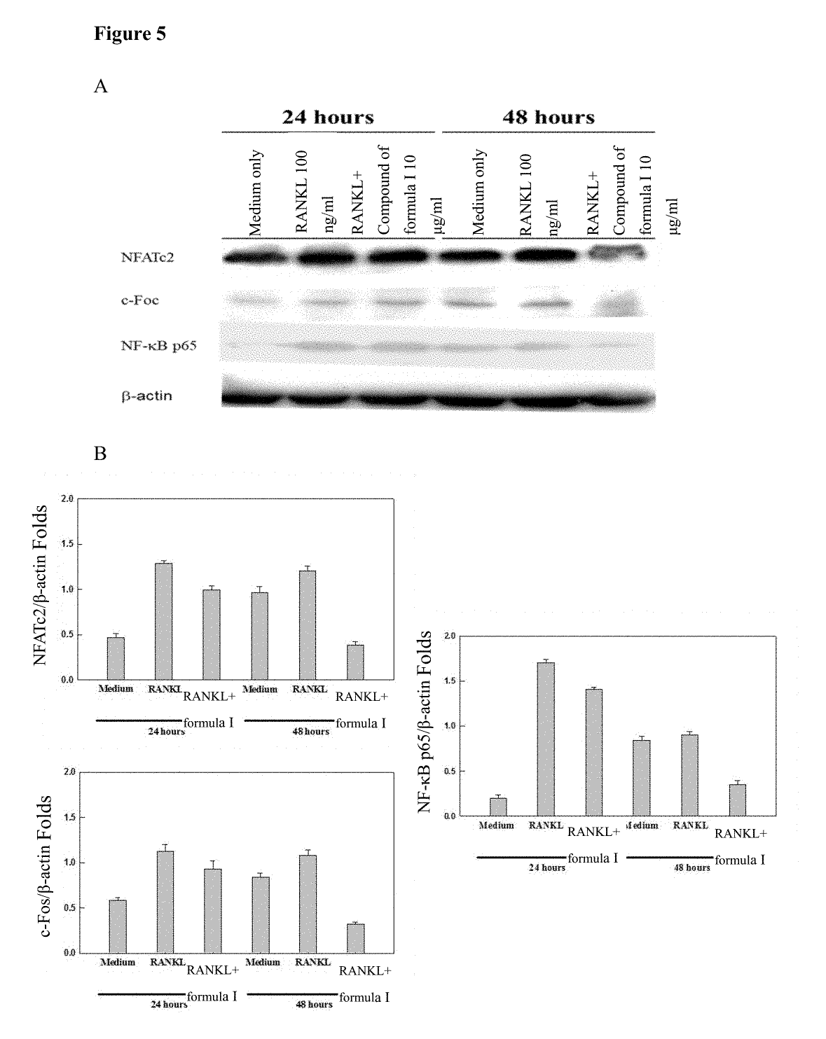

The effect of the compound of formula I on activity changes of the MAPK family downstream proteins and NF-κB in osteoclasts were studied. According to the conditions selected in a literature “Bone 2011, 48:1336-1345,” RAW 264.7 cells were treated with 100 ng/ml receptor activator of nuclear factor κB ligand or co-treated with 10 μg/ml of the compound of formula I for 24 hours, 48 hours, proteins were collected to analyze the expression of the MAPK family downstream proteins c-Fos and NFATc2 and NF-κB p65 by using the Western blot method. Downstream proteins c-Fos and NFATc2 were regulated by ERK, p38, JNK, were also co-treated with receptor activator of nuclear factor κB ligand and the compound of formula I for 48 hours, protein expression was significantly reduced due to inhibition (FIG. 5A, B). In addition, the compound of formula I would reduce NF-κB protein expression. In the receptor activator of nuclear factor κB ligand-induced osteoblast differentiation platform, NF-κB p65 was affected by the compound of formula I, protein expression was reduced at 48 hours (FIG. 5A, B). According to the above-described protein expression results, by inhibiting MAPK family proteins or NF-κB pathway the compound of formula I reduced the number of mature osteoclasts induced by receptor activator of nuclear factor κB ligand.

Effect of the compound of formula I on receptor activator of nuclear factor κB ligand-induced osteocytic activity of the osteoclast cells Osteoclast is a multinuclear cell, which differentiates because of the stimulation of macrophage colony stimulating factor and receptor activator of nuclear factor κB ligand, after differentiation the folds on the edge of the osteoclast form a resorption groove on the cell membrane and the bone surface is mineralized. In this space, the osteoblast secretes some enzymes and acids. These acids activate the enzymes to initiate osteolysis of bone mineral matrix. In the present invention, we purchased a corning osteo assay surface culture plate, RAW 264.7 cells were treated with receptor activator of nuclear factor κB ligand or co-treated with the compound of formula I for 12 days, during the process the culture solution was replaced with fresh ones of the same condition once every 3 days, osteoclast differentiation and groove formation were then observed.

In order to confirm the effect of the compound of formula I on the bone resorption capability of differentiated osteoclasts, RAW 264.7 cells were cultured in a 24-well plate (osteo assay plate, 24 well; corning incorporated) containing artificial bone material, the cell number of each well was 2×10³ cell/ml. The culture solution contained 100 ng/ml of receptor activator of nuclear factor κB ligand (for RAW 264.7; Peprotech) or co-treated with 10 μg/ml of the compound of formula I. The culture solution was replaced with fresh one (containing the above-mentioned treating drugs) every three days, after being cultured for 12 days, each well was washed with PBS and reacted with 5% sodium hypochlorite for 5 minutes, the cells were then removed from the plate. The number of grooves per each well was counted under a microscope 40× and further photographed for observation.

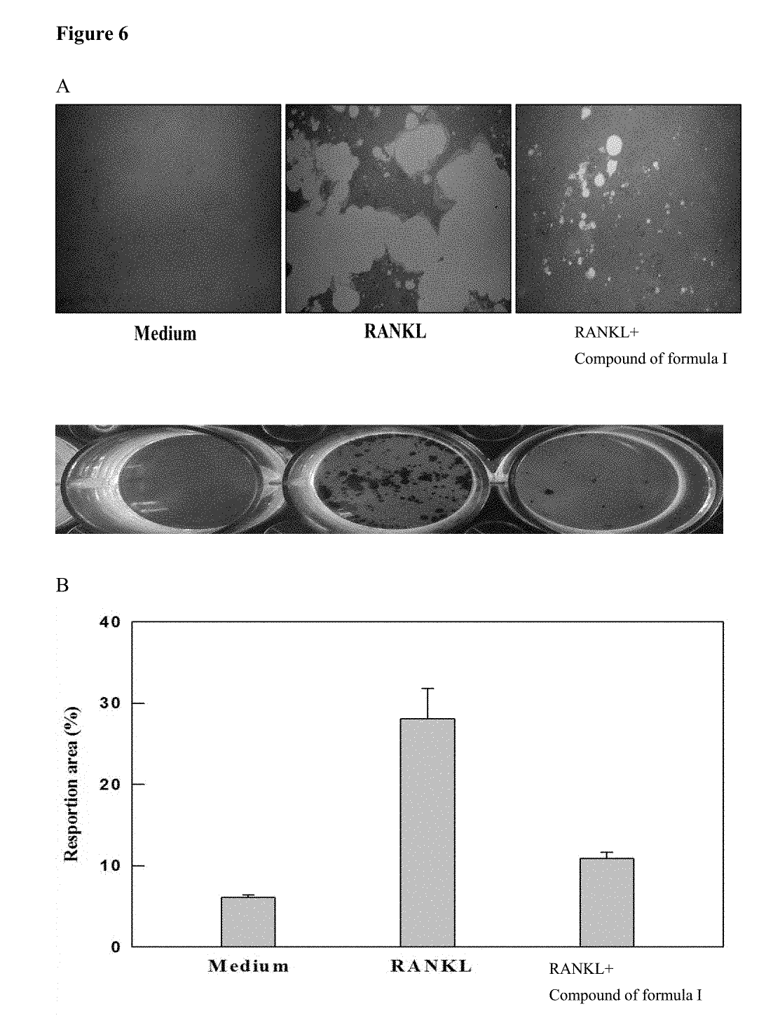

Under the microscope, each bright spot showed the capability of osteoclast cells to erode artificial bone material. Under the co-treatment condition of receptor activator of nuclear factor κB ligand and the compound of formula I, we were able to see that the osteolytic activity of osteoclast cells was inhibited by the compound of formula I, the eroded area of the artificial bone material was substantially reduced (FIG. 6A, B). These results showed that the compound of formula I not only reduced the osteoclast formation but also inhibited the osteolytic activity of osteoclast cells.

### Animal Experiment

Inducing osteoporosis animal models and experimental methods:
1. 6-month old Sprague-Dawley (SD) female rats were purchased;
2. These rats were divided into 7 groups, of which Group 1 was given pseudo-surgery, Group 2-7 underwent bilateral ovariectomies;
3. On the 6^{th} day after surgery, 1 mL/kg of vehicle alone was given orally to Group 1, Group 2 daily, Zoledronic Acid and Prolia were administered weekly by injections to Group 3, Group 4, respectively; 1, 3, 10 mg/kg of the compound of formula I was given orally every 3 days to Groups 5-7, respectively;
4. Body weight and bone density were measured weekly, blood was drawn until the 118th day;
5. Rats were then sacrificed to obtain kidneys, livers and rat tibia;
6. Data were consolidated and processed.

Experimental groups are shown in Table 1

TABLE 1
Experimental groups

| | | | | Number | | | |
|---|---|---|---|---|---|---|---|
| Group | Surgery | Drug | Route | of rats | dose | | Frequency |
| 1 | Pseudo S. | Vehicle alone | PO | 2 | 1 | mL/kg | Once daily |
| 2 | bilateral | Vehicle alone | PO | 3 | 1 | mL/kg | Once daily |
| | ovariectomy | | | | | | |
| 3 | bilateral | Zoledronic | IV | 2 | 0.67 | mg/kg | Once weekly |
| | ovariectomy | Acid | | | | | |
| 4 | bilateral | Prolia | ID | 2 | 10 | mg/kg | Once weekly |
| | ovariectomy | | | | | | |
| 5 | bilateral | Compound of | PO | 2 | 1 | mg/kg | Once every 3 days |
| | ovariectomy | formula I | | | | | |
| 6 | bilateral | Compound of | PO | 2 | 3 | mg/kg | Once every 3 days |
| | ovariectomy | formula I | | | | | |
| 7 | bilateral | Compound of | PO | 2 | 10 | mg/kg | Once every 3 days |
| | ovariectomy | formula I | | | | | |

The vehicle was ethanol:PEG 400:physiological saline = 10:20:70 (v/v/v)

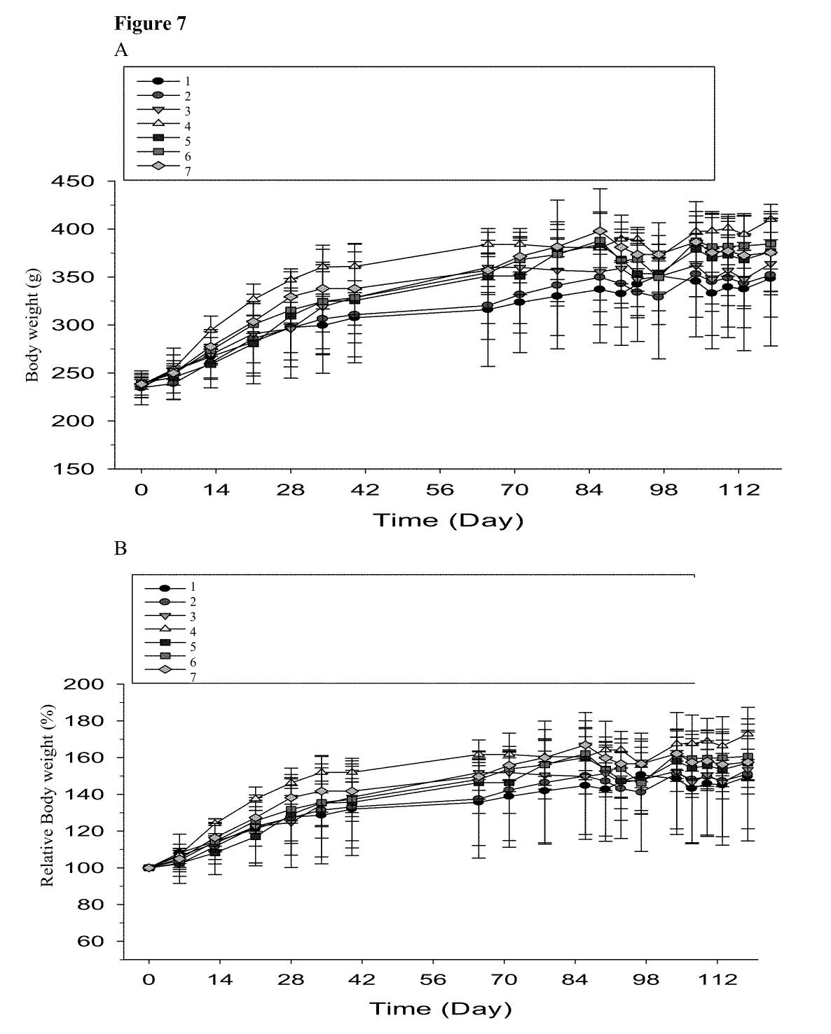

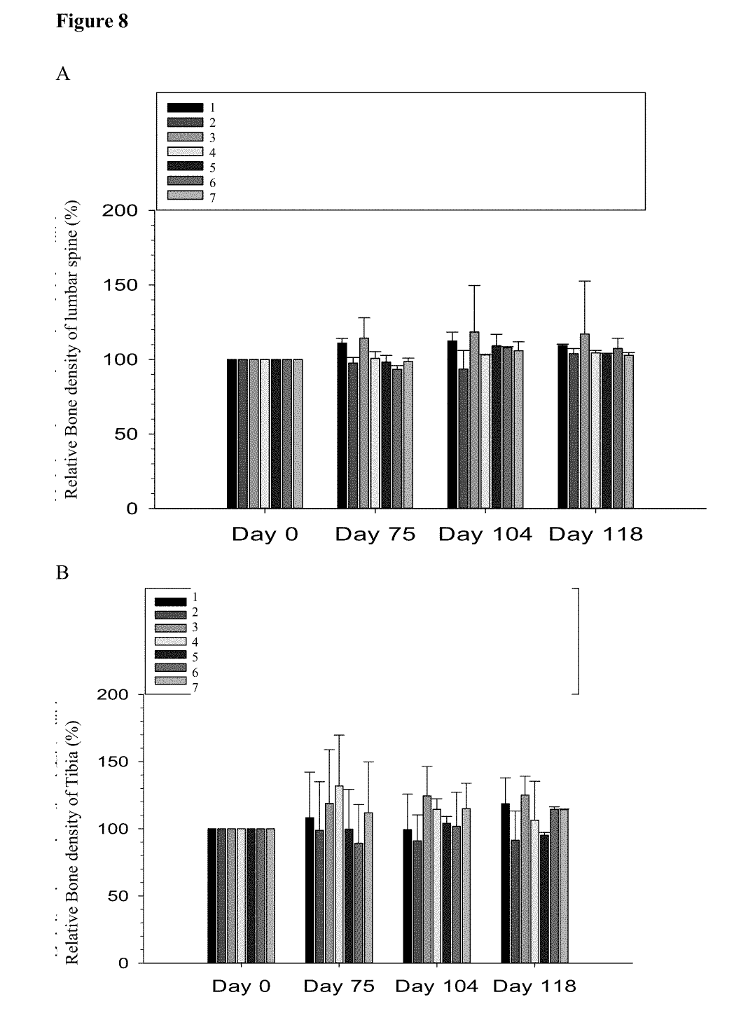

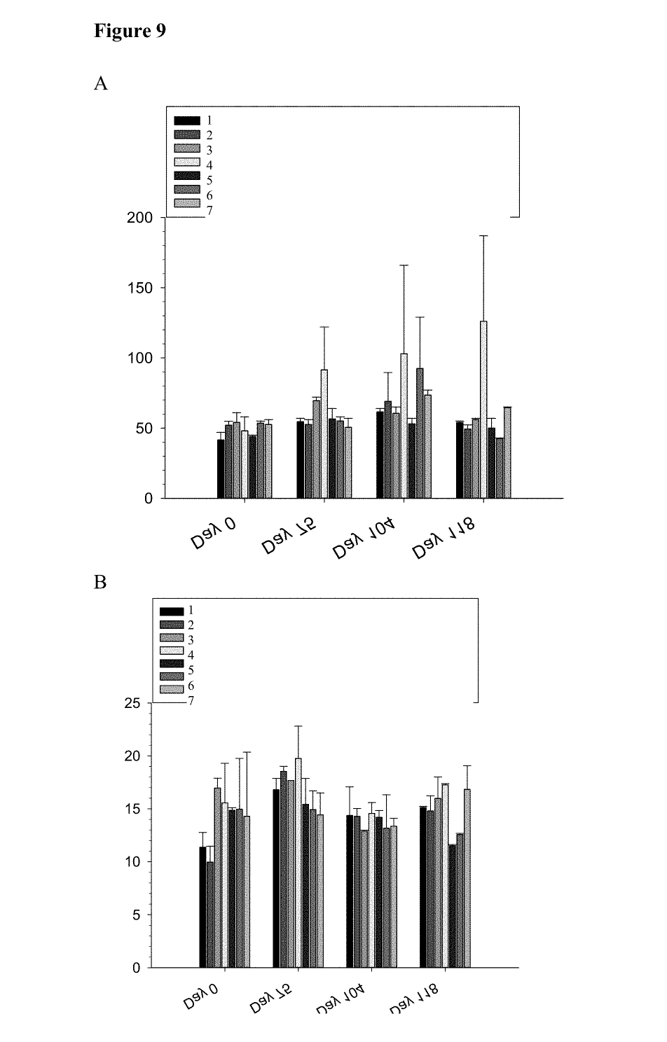

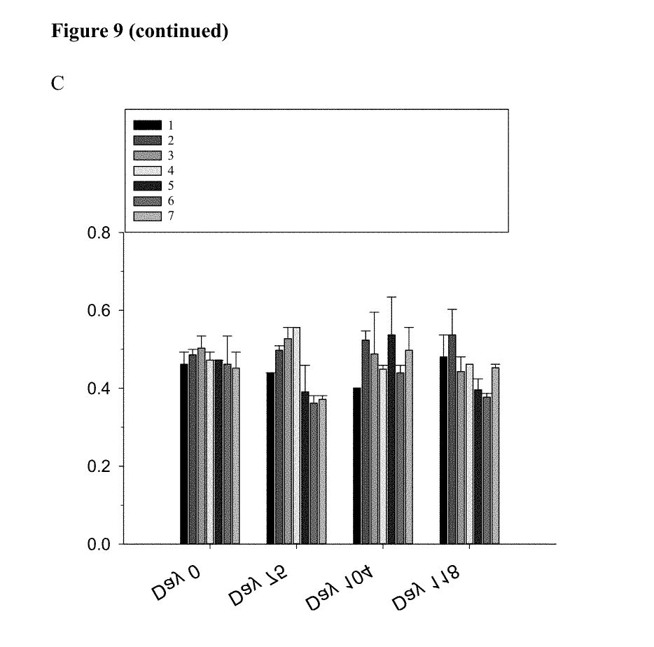

The experimental results are shown in FIGS. 7-9. FIG. 7 shows the effects of various treatments on the body weight of rats, A is the body weight of rats, and B is the relative body weight of rats; FIG. 8 shows the effects of various treatments on the bone density of lumbar vertebrae and tibial bone of rats, A was the relative bone density of the lower lumbar spine (3rd to 5th lumbar vertebrae), B was the relative bone density of the tibia, the results showed that the compound of formula I was able to increase the tibial bone density by 10% (as compared to the experimental group); FIG. 9 shows the effects of various treatments on liver and kidney toxicity in rats, A is the effects of various treatments on alanine aminotransferase in rats; B is the effects of various treatments on the blood urea nitrogen in rats; and C is the effects of various treatments on plasma creatinine in rats.

Results: In this experiment, we measured the therapeutic effects of various doses of the compound of formula I on the bone density of lower lumbar spine and tibial bone of ovariectomized rats, 19 weeks after ovarian removal various doses of the compound of formula I all were able to improve the reduced tibial bone density caused by ovarian removal (FIG. 8B). Animal experiments also showed that the compound of formula I was different from commercially available drugs, Proliaor and Zoledronic acid which would significantly affect the body weight of mice (Proliaor increased the body weight, Zoledronic acid decreased the body weight) (FIG. 7). The compound of formula I was able to reduce cytotoxicity in liver and kidney as compared to other commercially available drugs (less cytotoxicity in liver than Proliaor and less cytotoxicity in kidney than Zoledronic acid) (FIG. 9).

It should be understood that although the present invention has been specifically disclosed by above-described examples, they are non-limiting. It will be readily apparent to a person skilled in the art that varying substitutions and modifications may be made to the invention disclosed herein without departing from the scope and spirit of the invention.

What is claimed is:

1. A method for treating osteoporosis in a subject in need thereof comprising administering to the subject an effective amount of a composition comprising a compound of formula I or pharmaceutically acceptable salts thereof, wherein the compound of formula I is as follows:

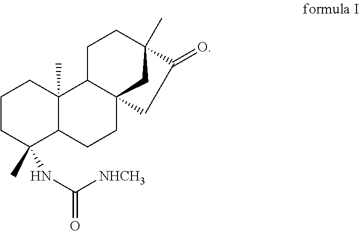 [SMILES: CNC(=O)N[C@]1(C)CCC[C@@]2(C)C3CC[C@@]4(C)C[C@]3(CCC21)CC4=O]

2. The method of claim 1, wherein the composition inhibits osteoclast differentiation.

3. The method of claim 1, wherein the composition inhibits osteolytic activity of osteoclasts.

4. The method of claim 1, wherein the dose of the compound of formula I is 1 mg/kg-400 mg/kg.

5. The method of claim 1, wherein the dose of the compound of formula I is 1 mg/kg-80 mg/kg.

6. The method of claim 1, wherein the dose of the compound of formula I is 1 mg/kg-25 mg/kg.

7. The method of claim 1, wherein the composition is an oral dosage form.
